(19) United States
(12) Patent Application Publication
(10) Pub. No.: US 2026/0055444 A1
(43) Pub. Date: Feb. 26, 2026

(54) POINT MUTATION RATE DETECTION METHOD

(71) Applicant: HITACHI HIGH-TECH CORPORATION, Tokyo (JP)

(72) Inventors: Hirokazu KATO, Tokyo (JP); Noriyuki SUMIDA, Tokyo (JP); Mitsuhiro MIYAZAKI, Tokyo (JP); Motohiro YAMAZAKI, Tokyo (JP)

(21) Appl. No.: 19/103,883

(22) PCT Filed: Sep. 22, 2022
(86) PCT No.: PCT/JP2022/035397

§ 371 Date: Feb. 14, 2025

(51) Int. Cl. C12Q 1/6844

(52) U.S. Cl. CPC C12Q 1/6844

(57) ABSTRACT

The point mutation rate detection method according to the present invention is used in a multiplex ligation-dependent probe amplification (MLPA) measurement, and includes: a measurement step of measuring at least an intensity S_{MT} among the intensity S_{MT} of a mutation-derived signal as a fluorescence signal emitted from a mutation site of a sample and an intensity S_{WT} of a wild-type derived signal as a fluorescence signal emitted from a site of the sample other than the mutation site, using an electrophoresis device; and a rate calculation step of calculating a rate of the intensity S_{MT} to a reference value having a higher intensity than the S_{MT}. In the point mutation rate detection method, an upper limit of a dynamic range of measurement for the fluorescence signal of the electrophoresis device is equal to or more than a predetermined value.

### TECHNICAL FIELD

This invention relates to a point mutation rate detection method.

### BACKGROUND ART

It is becoming clear that out of the 20,000 or so genes that exist, there are only a few hundred cancer genes and cancer suppression genes. Even now, gene mutations that cause cancer in humans are being discovered one after another, but it is thought that the number will eventually probably remain at around several hundred. In fact, the number of genes included in the OncoGuide NCC OncoPanel for next-generation sequencers, which was jointly developed by the National Cancer Center Japan and Sysmex and was approved for insurance coverage in 2019, is 124. The number of genes in Foundation-One CDx, developed by Foundation Medicine in the United States, is 324. These figures show that cancer diagnosis is being promoted by detecting the behavior of a limited number of genes in the hundreds. Of these genes, point mutations are particularly important. The reason for this is that cancer develops and progresses through accumulation of random point mutations.

On the other hand, these tests use a method called “massively parallelly” to decipher genes, that is a next-generation sequencer, and so the cost is inevitably high. In fact, the cost of the two cancer gene panel tests described above is 560,000 yen, which is a significant burden for ordinary patients.

Therefore, there is a need for technology that can measure a limited number of genes (around 100) quickly, accurately and at low cost. With current qPCRs, the number of genes that can be measured in a single tube is limited to the multicolorization of fluorescent dyes, and at most five colors, or five genes, is the limit. On the other hand, the next-generation sequencers are expensive. On the other hand, a capillary sequencer makes it possible to separate and detect PCR products according to their molecular weights by performing electrophoresis after the PCR. Therefore, it is possible to obtain information on the “length of the DNA molecule”that is not available with conventional qPCR.

The characteristics of this capillary sequencer are used in short tandem repeat analysis for human identification and the Multiplex Ligation-dependent Probe Amplification (MLPA) Method. In both of these techniques, molecular length information is extracted from the amplified products after a PCR amplification using a capillary electrophoresis, and then added to the data, allowing for judgments and diagnoses that cannot be achieved with PCR alone. In the case of the human identification, “separation by DNA molecule length” is performed by focusing on the short tandem repeat sequence, which is unique to genomic DNA molecules, while the MLPA methods actively add PCR probes of different lengths from the outside, thereby artificially performing “separation by DNA molecule length” from the outside. The former uses an intrinsic characteristics of biological samples, while the latter contrasts with this in that humans perform the molecular design from the outside.

The MLPA Method is a technique developed by Shouten in the Netherlands, which involves performing PCR with probes of varying lengths and then developing the PCR products using the capillary electrophoresis. Patent Literature 1 describes the basic technology of the MLPA Method. In addition, the company MRC Holland, which was established by Shouten, sells reagents that have been made into kits for the MLPA Method. The MLPA methods can detect copy number changes (deletions/duplications), DNA methylation, gene expression, and point mutations, which are most important for cancer diagnosis. Specific details of the point mutation detection using MLPA methods are described in Nonpatent Literature 1. Nonpatent Literature 2 is a product description (user manual) for a commercially available MLPA kit that can detect point mutations in eight genes, including JAK2, which are factors in myeloproliferative neoplasms, with high sensitivity of 1 to 5%. In this document, it is noted that the kit cannot be used for quantitative point mutation counting, and should only be used for qualitative point mutation counting. By qualitative, it means that the signal is compared with the control binning DNA that comes with the kit, and when the signal is greater than the binning DNA, it is judged that “mutations are present.” When the signal is smaller, it is judged that “no mutations are present.”

In order to detect the presence or absence of mutations, it is necessary to change a length of a stuffer sequence contained in the Left Probe Oligonucleotide (LPO) on the left of the two probes that hybridize to the target sequence in the MLPA methods. This is because the LPO is responsible for detecting a mutation status, and it is necessary to convert this mutation status into molecular length information in an electrophoresis.

There are also kits on the market that can detect two types of single nucleotide substitutions with different sequences for one point mutation, and these kits are described in Nonpatent Literature 3. Specifically, for the gene IDH2, the Wild Type is guanine. In contrast, a probe of 151 base length is assigned for point mutation adenine, and a probe of 145 base length is assigned for point mutation thymine. In other words, it can be seen that the length of the LPO has been changed in the commercially available kit.

On the other hand, the kits shown in these Nonpatent Literatures 2 and 3 do not include a probe for checking a Wild Type status. Generally, the point mutations occur in only a small part of a cell population. Generally, the rate MT/WT of cells (Mutant Type) that have point mutations to cells that have Wild Type as a normal state of no point mutations is important information for cancer diagnosis. However, since conventional MLPA Kits do not have a probe for Wild Type, it is not possible to confirm the MT/WT rate. The reason for this is that the signal detected for 1% point mutations is around 3,000 [ADU]. The point mutation of 1% means that 1% Mutant Type and 99% Wild Type are mixed together. In this case, when a signal is detected from the Wild Type probe, the signal from the Wild Type will be around 300,000 [ADU]. However, this signal volume exceeds the upper saturation limit of 32,767 [ADU] that can be detected by a conventional capillary sequencer, and so it cannot be measured. This is the reason why there are no probes for measuring Wild Type in the MLPA methods used for point mutation detection. In other words, the reason why there are no probes of Wild Type is due to the narrow dynamic range of the device.

On the other hand, Nonpatent Literature 4 reports a technique that can detect the rate MT/WT of Mutant Type to Wild Type up to 0.01% by expanding the dynamic range of the conventional capillary sequencer. The mutation detection limit of fragment analysis in the conventional capillary sequencer is said to be 1% to 5%. The reason for this is that the dynamic range of the conventional capillary sequencers is narrower than three orders of magnitude. Nonpatent Literature 4 reports on a technology that expands the dynamic range from equal to or more than three orders of magnitude to four orders of magnitude. In this patent, the capillary electrophoresis analysis technology that expands the dynamic range to equal to or more than three orders of magnitude is called HiDy.

### CITATION LIST

### Patent Literature

Patent Literature 1: International Publication WO 2001/61033

### Nonpatent Literature

Nonpatent Literature 1: Multiplex ligation-dependent probe amplification (MLPA) in tumor diagnostics and prognostics, Diagn Mol Pathol. 2012 December; 21(4): P. 189-206. doi: 10.1097/PDM.0b013e3182595516.
Nonpatent Literature 2: Product Description SALSA MLPA Probemix P520-A2 MPN mix 2 (Product description version A2-03; Issued 13 Jul. 2022), [Search date 2022.09.08], Internet <URL: https://www mrcholland.com/products/32322/Product %20des cription%20P520-A2%20MPN%20mix%202-v03.pdf>
Nonpatent Literature 3: Product description ME012-A1 MGMT-IDH1-IDH2 (Product Description version A1-03; Issued 3 Aug. 2021), [Search date 2022.09.08], Internet <URL: https://www. mrcholland.com/products/30043/Product %20des cription%20ME012-A1%20MGMT-IDH1-IDH2-v03.pdf>
Nonpatent Literature 4: Highly sensitive mutation quantification by highdynamic-range capillary-array electrophoresis (HiDy CE), Lab Chip, 2020, 20, P. 1083-1091

### SUMMARY OF INVENTION

### Technical Problem

The MLPA Method, which is one of the representative applications of the capillary electrophoresis, allows multiplexing of 40 probes. However, in the point mutation counting using the conventional MLPA methods, the presence or absence of mutations (MT) could be determined by comparing the signal intensities from the genes in the control sample provided with the kit and the target sample, but it was not possible to measure the rate. In other words, the point mutation counting using the conventional MLPA methods could perform the qualitative testing, but not the quantitative testing.

The present invention was made in light of the above situation. The present invention is intended to provide a point mutation rate detection method that can perform quantitative testing.

### Solution to Problem

A point mutation rate detection method according to the present invention that solves the above-described problem is used in a multiplex ligation-dependent probe amplification (MLPA) measurement. The point mutation rate detection method includes: a measurement step of measuring at least an intensity S_{MT} among the intensity S_{MT} of a mutation-derived signal as a fluorescence signal emitted from a mutation site of a sample and an intensity S_{WT} of a wild-type derived signal as a fluorescence signal emitted from a site of the sample other than the mutation site, using an electrophoresis device; and a rate calculation step of calculating a rate of the S_{MT} to a reference value having a higher intensity than the S_{MT}. An upper limit of a dynamic range of the measurement for the fluorescence signal of the electrophoresis device is equal to or more than a predetermined value. The reference value is a maximum saturation value of the S_{MT} measured independently of the measurement step in advance, and the rate calculation step calculates the rate of the S_{MT} obtained in the measurement step from the maximum saturation value.

### Advantageous Effects of Invention

The present invention is to provide a point mutation rate detection method that can perform quantitative testing.

Objects, configurations, and effects other than the above will be apparent from the description of the following embodiments.

### BRIEF DESCRIPTION OF DRAWINGS

FIG. 1 is an analysis reaction diagram describing one example of a method for detecting point mutation in DNA base sequence.

FIG. 2 is an analysis reaction diagram for a first example of the present invention.

FIG. 3 is an analysis reaction diagram of a second example of the present invention.

FIG. 4 is an analysis reaction diagram of a third example of the present invention.

FIG. 5 is an analysis reaction diagram of a fourth example of the present invention.

FIG. 6 is an analysis reaction diagram of a fifth example of the present invention.

FIG. 7 is an analysis reaction diagram of a sixth example of the present invention.

FIG. 8 is an analysis reaction diagram of a seventh example of the present invention.

FIG. 9 is a diagram showing analytical reaction analysis results for an eighth example of the present invention.

### DESCRIPTION OF EMBODIMENTS

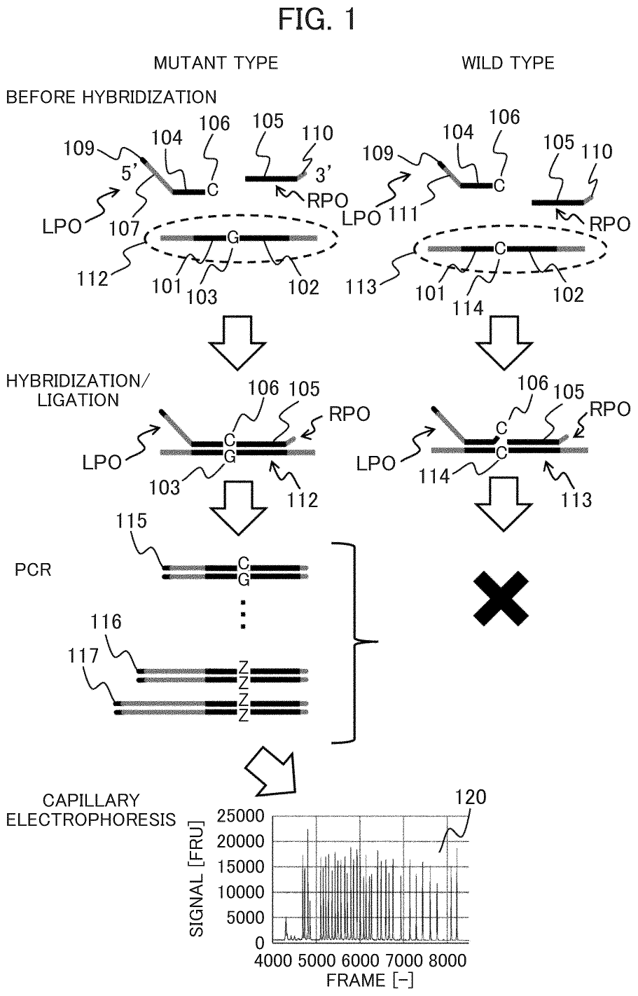

Hereinafter, embodiments of the present invention will now be described with reference to FIG. 1 to FIG. 9.

First, one example of a method for detecting point mutations in a DNA base sequence will be described using FIG. 1. FIG. 1 is an analysis reaction diagram that describes one example of a method for detecting point mutations in the DNA base sequence.

The method shown in FIG. 1 is a technique for multiplex detection of multiple fragments using a capillary electrophoresis for multiple point mutations. This method is generally referred to as Multiplex Ligation-dependent Probe Amplification (MLPA).

The method shown in FIG. 1 relates to a method for detecting point mutations in the DNA base sequence.

As shown before a hybridization in FIG. 1, at this stage, DNA target sequences 112 and 113, which are desired to investigate for point mutation status, and a Left Probe Oligonucleotide (LPO) and a Right Probe Oligonucleotide (RPO) are not hybridized. The DNA target sequences 112 and 113 are composed of bonded molecules of the four types of nucleotides: guanine, adenine, cytosine, and thymine. The DNA target sequence 112 has a point mutation guanine 103, which is a point mutation of 1 base pair. On the other hand, the DNA target sequence 113 is a Wild Type (Wild Type) with normal genes, and has a normal base cytosine 114, which is a correct sequence. A difference between the DNA target sequence 112 and the DNA target sequence 113 is only one base difference between the point mutation guanine 103 and the normal base cytosine 114, and surrounding sequences 101 and 102 located upstream and downstream of the point mutation guanine 103 are the same sequence.

As shown in FIG. 1 before hybridization described above, the LPO has a base cytosine 106 that matches the point mutation guanine 103 in the DNA target sequence 112. The LPO has a sequence 104 that is complementary to the sequence 101, and a stuffer sequence 107, 111 to adjust a length of the probe. The stuffer sequence 107 does not hybridize with the DNA target sequences 112 and 113. In this method, after the hybridization of these sequences, a ligation reaction is performed using a ligase enzyme to ligate the LPO and the RPO, and then the product is amplified by PCR. The LPO has a primer sequence 109 that is complementary to the PCR primer used for amplification by PCR.

As shown before hybridization in the above-described FIG. 1, the RPO has a sequence 105 that hybridizes in a complementary manner with the sequence 102. Furthermore, the RPO has a primer sequence 110 that is complementary to the PCR primer for amplifying by PCR as described above.

In the normal MLPA Method, a stuffer sequence 111 is inserted between the sequence 105 in the RPO and the primer sequence 110 in order to adjust the length of the PCR product. However, in order to detect the point mutations, it is preferable to insert the stuffer sequence 107 into the LPO, as shown in FIG. 1. The reason for this is that it is the LPO that is substantially detecting the point mutations. In order to convert this point mutation information into the base length, it is absolutely necessary to arrange the stuffer sequence 107 in the LPO. In other words, even when the stuffer sequence 107 is present in the RPO, it is difficult to detect the point mutations as base length.

Next, as shown in the hybridization/ligation in FIG. 1, the Left Probe Oligonucleotide (LPO) and the Right Probe Oligonucleotide (RPO) are hybridized to the DNA target sequences 112 and 113, which are the targets of hybridization.

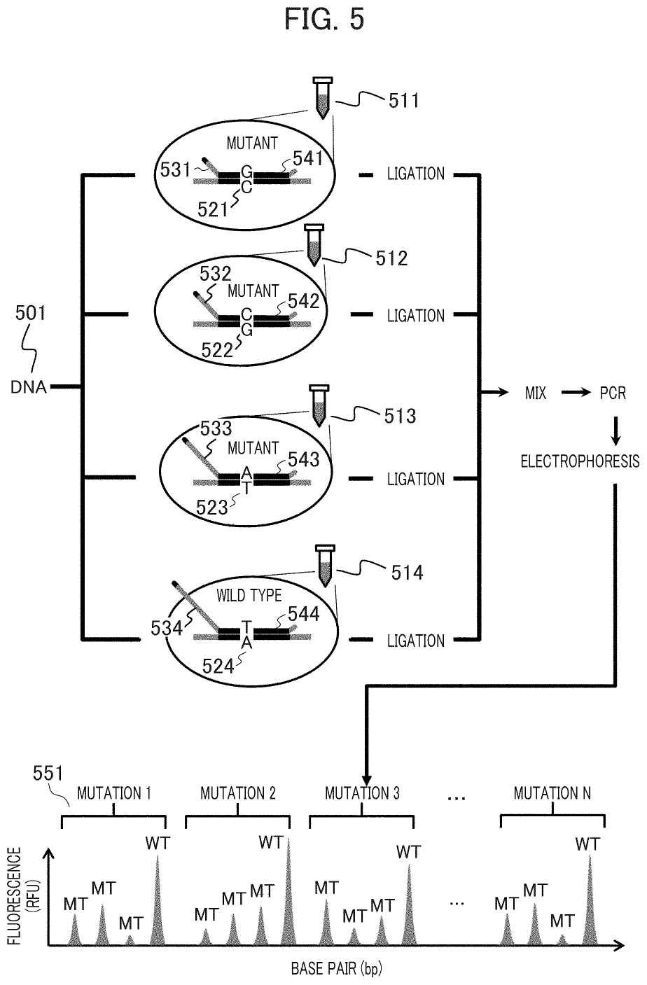

For example, as shown in the hybridization/ligation in FIGS. 1, 5 μL of 10 mM Tris buffer (pH 8.0) containing 50 to 100 ng of the DNA target sequences 112 and 113 is heated at 98°C. for 5 minutes, then cooled to room temperature, and the LPO and the RPO are added. By incubating at 60° C. for 18 hours, the DNA target sequence 112 hybridizes with the LPO and the RPO. In addition, the DNA target sequence 113 hybridizes with the LPO and the RPO.

Here, the cytosine 106 at the 3′ end of the LPO hybridizes with the point mutation guanine 103 because they are complementary. After the hybridization, the cytosine 106 as the 3′ end of the LPO and the 5′end of the sequence 105 in the RPO are adjacent, so the ligase enzyme can ligate them. In other words, in the Mutant Type, the LPO and the RPO can be made into a single DNA strand. On the other hand, in the case of the Wild Type, the DNA target sequence 113 has the normal base cytosine 114, so it cannot form a complementary strand with the cytosine 106 at the 3′ end of the LPO. Therefore, the ligase enzyme cannot ligate the LPO and the RPO. In other words, in the Wild Type, the LPO and the RPO cannot form a single DNA strand.

Next, it moves on to the PCR step shown in FIG. 1. In the PCR step, the DNA target sequence 112 and the DNA strand that has become a single strand as a result of the LPO and the RPO being ligated by a ligase reaction are denatured at a temperature of 94° C. In addition, the DNA target sequence 113, and the LPO and the RPO are denatured at a temperature of 94°C.

The primers added to the solution after the denaturation hybridize to the primer sequences 109 and 110 of the single-stranded DNA that has been denatured, and by repeating the heat cycle, it is possible to amplify the specified DNA fragment exponentially. It is important to note that only the mutant-type DNA fragments in which the LPO and the RPO are ligated by hybridization/ligation in FIG. 1 are amplified by PCR, and the wild-type DNA fragments are not amplified. In other words, when the DNA target sequence 113 has the normal base cytosine 114, the LPO and the RPO will not be ligated and will not be amplified, and as a result, no PCR product will be generated.

The above-described reaction describes one point mutation in the genome in detail. This reaction can be performed in parallel (that is, multiplexed) to detect the multiple point mutations present in the genome. In fact, the MLPA reaction can detect equal to or more than 40 different probes at once in Copy Number Variation and methylation detection. Therefore, it is possible to apply the MLPA reaction to the point mutation detection and detect equal to or more than 40 different probes at once.

Next, as shown in the capillary electrophoresis (CE) in FIG. 1, the 40 DNA fragments as the PCR products produced after the PCR reaction are subjected to the electrophoresis using the capillary sequencer. In the base lengths of the generated PCR products, since the lengths of the LPO stuffer sequences 107 and 111 applied to respective point mutations are changed, the base lengths of PCR products 115, 116, and 117 can be changed to any length. In one example shown in FIG. 1, the base length to be changed was set to 15 bases. The difference in base length of the stuffer sequences is not limited to 15 bases, and can be changed from 1 to 100 bases.

In one example described in FIG. 1, the DNA target sequence 112 with a Mutant Type mutation is amplified, while the DNA target sequence 113 with a Wild Type mutation is not amplified. Therefore, the PCR products 115, 116, and 117 amplified from the multiple DNA target sequences 112 with mutations are developed in the capillary. The molecular lengths of the PCR products 115, 116 and 117 are different because their respective stuffer sequences are different, and so they have different molecular lengths similarly. Therefore, they are separated in the capillary electrophoresis, and signals corresponding to different times are detected. This is expressed in an electropherogram 120.

In one example alone described in FIG. 1, the presence or absence of mutations can be confirmed, but it is not possible to perform a quantitative test. In this embodiment, in order to perform the quantitative test, a rate of an intensity S_{MT} to the reference value with a higher intensity than the intensity S_{MT} of the mutation-derived signal, which is the fluorescence signal emitted from the mutation site of the sample, is calculated.

One example that embodies this is a point mutation rate detection method that includes a measurement step and a rate calculation step can be cited.

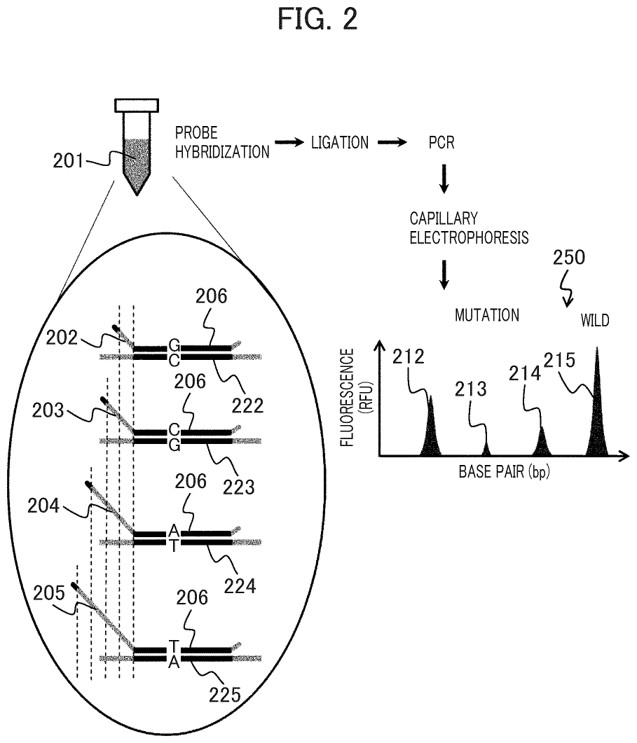

Here, the measurement step is a step of measuring at least an intensity S_{MT} among the intensity S_{MT} of a mutation-derived signal as a fluorescence signal emitted from a mutation site of a sample and an intensity S_{WT} of a wild-type derived signal as a fluorescence signal emitted from a site of the sample other than the mutation site, using an electrophoresis device. This measurement step can be performed using the capillary electrophoresis, as described in FIG. 1, or using an example described in FIG. 2 and subsequent figures.

In addition, the rate calculation step is a step of calculating the rate of the above-described S_{MT} to the reference value with a higher intensity than the above-described S_{MT}. The above-described reference value includes, for example, the intensity S_{WT} of the above-described wild-type derived signal and the maximum saturation value of the Mutant Type. Furthermore, since the above-described reference value has a very high signal intensity, the upper limit of the dynamic range for measurement of the fluorescence signal of the electrophoresis device is set to equal to or more than a predetermined value.

In this way, the point mutation rate detection method can calculate the relative rate of the intensity S_{MT} of the mutation-derived signal, and thus perform the quantitative testing.

The maximum saturation value of the Mutant Type is the intensity of the fluorescence signal obtained when the cell rate is 100% Mutant Type-derived (mutation-derived). It would be good to measure the above-described maximum saturation value in advance using the cultured cells cloned from living cells of the Mutant Type that has the corresponding point mutation. In other words, it would be good to measure the maximum saturation value (the reference value) in advance independently of the measurement step described above.

The upper limit of the dynamic range can be, for example, 200,000 [ADU] or more, 400,000 [ADU] or more, 600,000 [ADU] or more, 800,000 [ADU] or more, or 1,000,000 [ADU] or more, but is not limited to these values.

Therefore, the dynamic range can be, for example, 0 to 200,000 [ADU], 0 to 400,000 [ADU], 0 to 600,000 [ADU], 0 to 800,000 [ADU], or 0 to 1,000,000 [ADU], but is not limited to these. The dynamic range can be set to, for example, 0 to 1,500,000 [ADU].

With this upper limit of the dynamic range and the dynamic range, even when the reference value with a higher intensity than the above-described S_{MT} is measured, the intensity is unlikely to saturate, thus allowing the more accurate quantitative inspection.

In the point mutation rate detection method of this embodiment, as one example of the above-described quantitative inspection method, the rate of Mutant Type and Wild Type can be calculated, as well as the rate of Mutant Type to the maximum saturation value. In order to calculate the rate of Mutant Type to Wild Type, it would be good to perform the PCR amplification on a DNA fragment that includes the DNA target sequence 113 of the Wild Type.

The following is a description of some examples of the point mutation rate detection method according to this embodiment that uses these methods.

FIG. 2 is an analysis reaction diagram according to a first example of the present invention. This example describes a technique that makes it possible to measure the amounts of these mutations and Wild Type, and to quantify the rate of Mutant Type to Wild Type when up to three types of mutations occur in relation to Wild Type in the case of point mutations occurring at a single location.

As shown in FIG. 2, a sample tube 201 contains DNA target sequences 222, 223, and 224 therein, which have point mutations at specific one location in the genome. In this example, the DNA target sequences 222, 223, and 224 contain mutations, and a DNA target sequence 225 is a wild-type sequence. The point mutations in each target sequence are cytosine, guanine, thymine, and adenine. As described with reference to FIG. 1, these four different types of LPOs 202, 203, 204, and 205, which can form complementary strands for each of these four different types of target sequence, are co-existed. The bases at the 5′ end of the LPOs 202, 203, 204, and 205 are guanine, cytosine, adenine, and thymine, respectively. In addition, among the stuffer sequences of these LPOs, the stuffer sequence of the LPO 202 is the shortest, and the stuffer sequence of the LPO 205 is the longest. The difference between the respective stuffer sequences is 15 bp, and the difference in base length between the largest and smallest is 45 bp. The reason that 15bp is suitable for the distance between the ligated probes is that when the difference is less than 15 bp, the peaks during electrophoresis will be close together, making it difficult to separate them. In particular, when the rate MT/WT of Mutant Type with respect to 1% or 0.1% of Wild Type is detected, it is desirable that the distance between the two is 15 bp or more. However, the difference in the stuffer sequence is not limited to 15 bp. The rate MT/WT of Mutant Type to Wild Type can be calculated appropriately by using an electrophoresis device to calculate the rate S_{MT}/S_{WT} of the intensity S_{MT} to the intensity S_{WT} from the intensity S_{MT} of the mutation-derived signal, which is a fluorescence signal emitted from the mutation site of the sample, and the intensity S_{WT} of the wild-type derived signal, which is a fluorescence signal emitted from a part of the sample other than the mutation site.

In addition, since a RPO 206 used for point mutation detection in one location in this reaction is the same for all, the PCR reaction can be performed using a single type of the RPO 206. Since the ligation reaction only proceeds when the 3′ end of the LPO is complementary to the point mutation sequence in the target sequence, the “point mutation” information can be converted into “length” information for the base length of the stuffer sequence.

It is worth noting here that the MLPA Method used for conventional point mutation detection only measures mutations in Mutation, and does not measure mutations in Wild Type. The MLPA Method used for conventional point mutation detection uses the signal from the control sample, which is said to contain 1% of the mutation amount and comes with the kit, as a reference. The signal volume of the control sample for a specific point mutation is compared with the signal volume from the sample to be measured, and when the signal volume from the sample is greater than the signal volume from the control sample, it is judged that “mutations are present,” and when it is less, it is judged that “no mutations are present.” In addition, the manual clearly states that the MLPA Method used for conventional point mutation detection can only refer to the presence or absence of mutations, and cannot quantify MT/WT (MRC Holland Product Description SALSA (Registered trademark) MLPA (Registered trademark) Probemix P520-A2 MPN mix 2).

The reason why MT/WT cannot be calculated is that, first and foremost, the LPO probe for WT is not included in the kit. The reason why the LPO probe for WT cannot be included in the kit is that, with conventional capillary sequencers, the measurement of the LPO probe for WT reaches the saturation value, and accurate measurement is not possible. In other words, this is because the dynamic range of conventional capillary sequencers is insufficient. In detail, while the maximum measured value (saturated measurement value) for signal volume on conventional capillary sequencers is 32,767 [ADU], the signal from the WT greatly exceeds this maximum measured value. In other words, since the actual saturation occurs, it is not possible to accurately obtain the signal value of the WT. This makes it difficult to measure MT/WT. To be more specific, since the signal for 1% MT is around 2,000 [ADU], the signal for 100% WT is expected to be around 200,000 [ADU]. However, the current saturation signal intensity for the capillary electrophoresis is 32,767 [ADU], thus, it is not possible to measure 100% WT.

In contrast, the method of the capillary electrophoresis measurement with a high dynamic range reported in recent papers has been reported to be able to expand the dynamic range. More specifically, the dynamic range, which was previously less than three digits, has been expanded from three digits or more to four digits. Therefore, when this technology is used, signals from derived Wild Type can be detected without saturation. Therefore, it is possible to calculate the rate of MT/WT. In this example, the capillary electrophoresis analysis technology that expands the dynamic range to three digits or more is called HiDy.

There are other advantages to being able to measure Wild Type. Another reason why it is not possible to quantify Mutant Types using conventional kits is that the sample loading amount into the capillary varies at the time of injection. In the conventional method, only Mutant Types are measured for one point mutation, thus, the signal value includes variation in the sample loading amount at the time of injection. However, when it is possible to inject both the Mutant Type and Wild Type into the same capillary at the same time of injection, it is possible to compensate for the injection variation and cancel out the variation in injection by calculating the MT/WT. In this example, since the Wild Type is measured and the MT/WT is calculated, more accurate measurements are possible, and the quantitative measurements can be made. In addition, in this example, even for samples with unknown point mutation status, by simultaneously detecting both the Mutant Type and Wild Type signals in a single electrophoresis, it is possible to quantitatively, easily, quickly and inexpensively detect the contamination rate of abnormal cells such as cancer cells.

In the electrophoresis, peaks 212, 213, 214, and 215 can be seen on an electropherogram 250. The respective peaks correspond to the DNA target sequences 222, 223, 224, and 225. Dividing the signal values of the mutation-derived peaks 212, 213, and 214 by the signal value of the peak 215, which corresponds to Wild Type, allows calculating the MT/T.

This example shows that it is possible to identify and quantify four different type of bases for one point mutation, but this does not impose any restrictions on the number of multiplexes for point mutations. As shown in FIG. 1, it is possible to multiplex the number of point mutations to be measured. Generally, in a fragment analysis using a capillary sequencer, the separation is performed in a range of 100 to 500 bp. When a stuffer sequence with a base interval of 15 bp is designed, the number of mutations that can be measured is (500-100) bp/15 bp. Thus, it is possible to measure and analyze about 25 mutations in a single electrophoresis. Furthermore, when the PCR primer sequences within the LPO and the RPO can be amplified using, for example, six different primer sets, multicolor analysis becomes possible. This makes it possible to collectively measure 25 mutations×6 colors=150 mutations in a single electrophoresis.

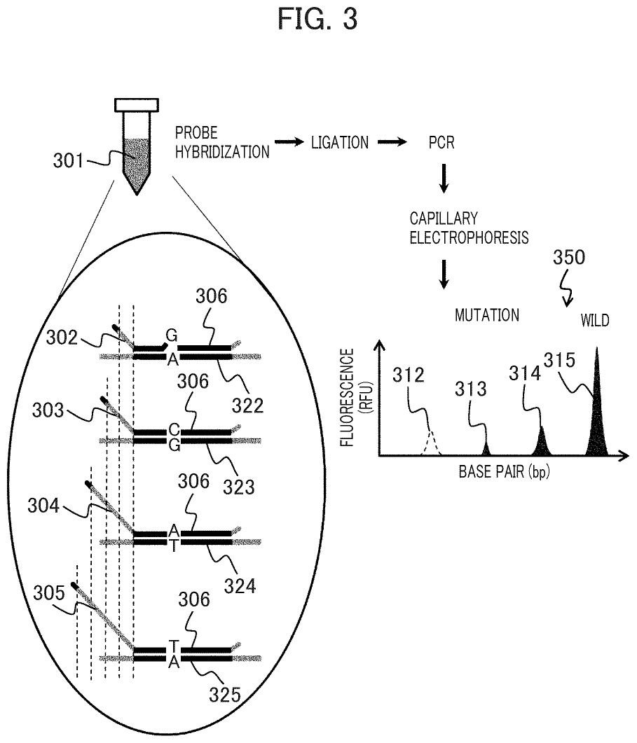

Next, referring to FIG. 3, a second example of the present invention will be described. FIG. 3 is an analysis reaction diagram of the second example of the present invention. This example differs from the first example in the following respects. In the first example, there were three types of point mutations in the DNA target sequence, whereas in the second example, there are two types of point mutations. Specifically, adenine is present in DNA target sequences 322 and 325, while point mutations of guanine are present in a DNA target sequence 323 and point mutations of thymine are present in a DNA target sequence 324. In addition, the DNA target sequences 325 and 322 are Wild Type, while the other DNA target sequences 324 and 323 are Mutant Type.

In the second example, similarly to the first example, four types of LPOs 302, 303, 304, and 305 and one type of a RPO 306 are added to a single sample tube 301 containing the mixed DNA target sequences. Although the LPO and the RPO hybridize with the DNA target sequence, the G at the 3′ end of the LPO 302 is not complementary to the point mutation A in the DNA target sequence 322. Thus, the hybridization cannot be achieved. Therefore, the 3′ end of the LPO 302 and the 5′ end of the RPO 306 cannot be ligated in the ligation reaction after the hybridization. Therefore, the PCR amplification derived from the DNA target sequence 322 does not proceed in the next step of the PCR reaction.

Therefore, in the fragment analysis of the capillary electrophoresis, the detected peaks are peaks 313, 314, and 315, which correspond to the DNA target sequences 323, 324, and 325, as shown in an electropherogram 350. A peak 312, which corresponds to a position of the DNA target sequence 322, is not detected. By comparing these peak values, it is possible to calculate the MT/WT for each mutation, as well as the presence or absence of mutations. Therefore, the quantitative measurements can be made.

This example describes the point mutation detection at a single location, as can be easily guessed, this method enables the multiplex detection of the multiple point mutations, and it is possible to collectively measure equal to or more than 40 mutations at once in a single electrophoresis.

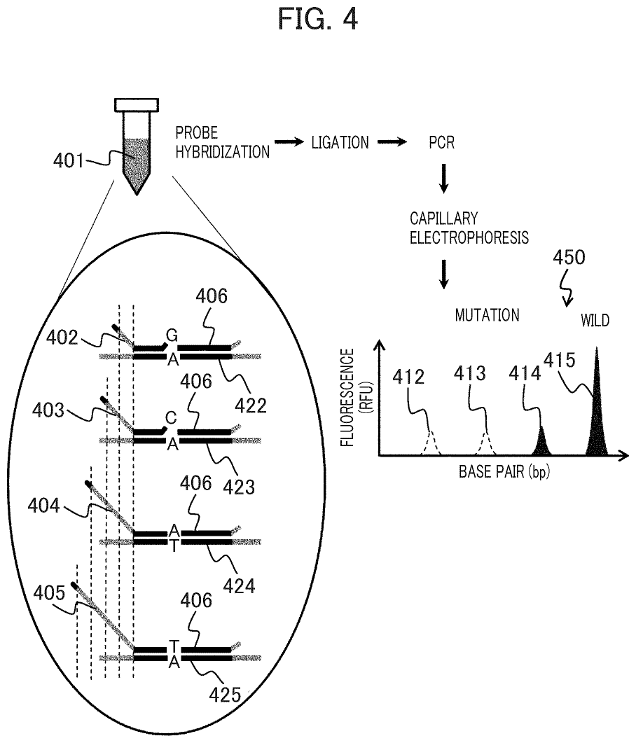

Next, referring to FIG. 4, a third example of the present invention will be described. FIG. 4 is an analysis reaction diagram of the third example of the present invention. This example differs from the first example in the following respects. In the first example, there were three types of point mutations in the DNA target sequence, whereas there is only one type of point mutation in the third example. Specifically, adenine is present in DNA target sequences 422, 423, and 425, whereas a DNA target sequence 424 has a point mutation of thymine. The DNA target sequences 422, 423, and 425 are Wild Type with adenine at the mutation site, and the other DNA target sequence 424 is Mutant Type with point mutation thymine.

In the third example, similarly to the first example, four types of LPOs 402, 403, 404, and 405 and one type of a RPO 406 are added to a single sample tube 401 containing the mixed DNA target sequence. Although the LPO and the RPO each hybridize with the DNA target sequence, the G at the 3′ end of the LPO 402 is not complementary to the point mutation A in the DNA target sequence 422. Thus, it cannot be completely hybridized. In addition, the C at the 3′end of the LPO 403 is not complementary to the point mutation A in the DNA target sequence 423. Thus, it cannot be completely hybridized.

Therefore, the 3′ end of the LPO and the 5′end of the RPO cannot be ligated in the ligation reaction after the hybridization in the DNA target sequences 422 and 423. Therefore, in the next step of the PCR reaction, the PCR amplification derived from the DNA target sequences 422 and 423 does not proceed.

Therefore, in the fragment analysis of the capillary electrophoresis, the detected peaks are peaks 414 and 415, which correspond to the DNA target sequences 424 and 425, as shown in an electropherogram 450. Peaks 412 and 413 at positions corresponding to the DNA target sequences 422 and 423 are not detected. By comparing these peak values, it is possible to calculate the MT/WT for each mutation, as well as the presence or absence of mutations. Therefore, the quantitative measurements can be made.

This example describes the point mutation detection at a single location, as can be easily guessed, this method enables the multiplex detection of the multiple point mutations, and it is possible to collectively measure equal to or more than 40 mutations at once in a single electrophoresis.

Next, referring to FIG. 5, a fourth example of the present invention will be described. FIG. 5 is an analysis reaction diagram of the fourth example of the present invention. This example differs from the first to fourth examples in that an initial DNA 501 is divided into four parts and added in equal amounts to four different sample tubes 511, 512, 513, 514 while the first to fourth examples performed ligation and hybridization reactions in a single sample tube. There is also a difference in that different LPOs 531, 532, 533, and 534 are added to these. The 3′ ends of the LPOs 531, 532, 533, and 534 shown in the figure are guanine, cytosine, adenine, and thymine, respectively. In addition, the same RPOs 541, 542, 543, and 544 are added to respective tubes. In this example, for the sake of simplicity, only one point mutation is shown in the diagram, but in actual reagents, there are as many sets of corresponding LPO and RPO as there are point mutations. In other words, there are as many LPO534s for Wild Type as there are wildtypes in the sample tube 514. Also, the 3′ end of the LPO 534 is not always thymine, and the LPO 534 is designed based on the sequence information of the Wild Type for each point mutation. In other words, when this method is applied, the peak for the DNA fragment derived from Wild Type for one point mutation becomes longer than the peaks derived from the other three DNA fragments (the intensity S_{WT} of the wild-type derived signal becomes higher). Also, in terms of elution time, the peak for the DNA fragment derived from Wild Type elutes the slowest compared with the other three peaks.

On the other hand, the remaining three LPOs 531, 532, and 533 are assigned point mutation information other than Wild Type. The sequence information for these point mutations differs from each other, and it is sufficient when they are in an exclusive state.

Alternatively, it is also useful to fix the 3′ ends of the LPOs 531, 532, 533, and 534 added to the sample tubes 511, 512, 513, and 514 to guanine, cytosine, adenine, and thymine, respectively, without considering the Wild Type status. It is useful to perform this fixation status for multiple point mutation groups.

By adjusting the length of the stuffer sequence, it is possible to design the base length of each probe. Therefore, the base length of the PCR product identified by electrophoresis can be easily sized, and it is possible to distinguish which peak is Wild Type and which peak is Mutant Type. It is possible to calculate the signals of point mutations and Wild Types from the assigned peaks and to quantify the MT/WT (quantify the rate S_{MT}/S_{WT} of the intensity S_{MT} of the mutation-derived signal and the intensity S_{WT} of the wild-type derived signal). In this example, for each point mutation, the LPO 534 corresponding to Wild Type has the longest stuffer sequence. This avoids the effects of sloping, where the signal intensity decreases as the base length increases. In other words, by grouping the electrophoresis for each point mutation, for example, when the difference in base length of the stuffer sequence is 15 bp, the difference in base length within the point mutation can be suppressed to 15 bp×(4 bases−1)=45 bp. This makes it possible to calculate the MT/WT more accurately. In addition, in order to make the detection regarding the low frequency point mutation more advantageous, the method of making the PCR fragments related to the mutation shorter and performing electrophoresis earlier is effective.

The advantage of this example over the first to third examples is that it avoids a competitive hybridization to the DNA target sequence that can occur between the LPO probes by dividing the LPO according to the base species at the 3′ end. This suppresses competition between the LPO probes and improves the reliability of the MT/WT values.

Since the 3′ ends of the LPOs 531, 532, 533, and 534 have guanine, cytosine, adenine, and thymine, respectively, they hybridize with cytosine, guanine, thymine, and adenine of DNA target sequences 521, 522, 523, and 524, which have been dispensed into respective sample tubes. In addition, the DNA target sequences 521, 522, 523, and 524 hybridize with the RPOs 541, 542, 543, and 544. The ligation is performed separately for the sample tubes 511, 512, 513, and 514. Subsequently, the sample tubes 511, 512, 513, and 514 are combined into one sample tube and the PCR is performed. After the PCR, each fragment can be separated according to its molecular weight in a single capillary by subjecting it to the capillary electrophoresis. Specifically, an electropherogram 551 shown in FIG. 5 can be obtained. As shown in the electropherogram 551, the MT/WT can be calculated for each Mutation 1, 2, 3 . . . N.

In this example, the PCR products are labeled with fluorescent labels using fluorescent primers during the PCR. These fluorescent primers are not limited to a single fluorescent dye, and it is also possible to label each sample tube with a different wavelength of fluorescent dye. It is also possible to design the probe groups within the LPO and the RPO such that each point mutation can be labeled with a different fluorescent dye.

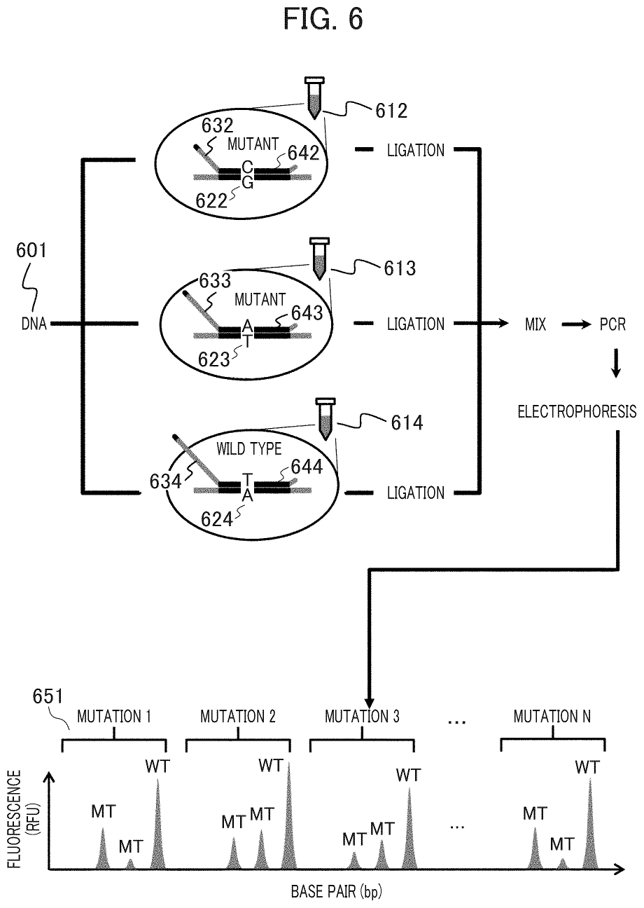

Next, referring to FIG. 6, a fifth example of the present invention will be described. FIG. 6 is an analysis reaction diagram of the fifth example of the present invention. Compared with the fourth example, this example is effective for reducing the number of sample tubes to be divided and reducing the cost required per electrophoresis. The point mutations do not necessarily occur in all three types of bases other than Wild Type, and in most cases, they can be limited to two or less types. In particular, cancers are highly diverse, and the point mutations that differ organ-specifically, such as lung cancer, breast cancer, and pancreatic cancer, occur. Therefore, it is very useful to increase the number of multiplexes that can be detected from a single capillary in a single electrophoresis performance by limiting the number of point mutations to be detected to two types or less.

In this example, an initial DNA 601 is divided into three parts and added in equal amount to three different sample tubes 612, 613, and 614. To these, different LPOs 632, 633, and 634 are added respectively. The 3′ ends of the LPOs 632, 633, and 634 shown in the figure are cytosine, adenine, and thymine, respectively. In addition, the same RPOs 642, 643, and 644 are added to respective tubes. In this example, for the sake of simplicity, only one point mutation is shown in the diagram, but in actual reagents, there are as many sets of corresponding LPO and RPO as there are point mutations. In other words, the number of LPO634s for Wild Type corresponding to the number of Wildtypes are present in the sample tube 614. In addition, the 3′end of the LPO 634 is not always thymine, and the LPO 634 is designed based on the sequence information of Wild Type for each point mutation. In other words, when this method is applied, the peak for the DNA fragment derived from Wild Type for one point mutation becomes longer than the peaks derived from the other three DNA fragments (the intensity S_{WT} of the wild-type derived signal becomes higher). Also, in terms of elution time, the peak for the DNA fragment derived from Wild Type is a fragment that elutes the slowest compared with the other three peaks.

On the other hand, the remaining two LPOs 632 and 633 are assigned point mutation information other than Wild Type. The information for each of these is different from the other, and it is sufficient when they are in an exclusive state. In the sample tubes 612, 613, and 614, the respective LPO and RPO hybridize with DNA target sequences 622, 623, and 624, respectively. The LPO and the RPO are ligated by ligase reaction and then amplified by PCR. In addition, the base length of each probe can be designed by adjusting the length of the stuffer sequence.

Therefore, the base length of the PCR product identified by electrophoresis can be easily sized, and it is possible to distinguish which peak is Wild Type and which peak is Mutant Type. Specifically, an electropherogram 651 shown in FIG. 6 can be obtained. As shown in the electropherogram 651, the MT/WT can be calculated for each Mutation 1, 2, 3 . . . N. In other words, it is possible to calculate the signals of point mutations and Wild Types from the assigned peaks and to quantify the MT/WT (quantify the rate S_{MT}/S_{WT} of the intensity S_{MT} of the mutation-derived signal to the intensity S_{WT} of the wild-type derived signal). In this example, for each point mutation, the LPO 634 corresponding to Wild Type has the longest stuffer sequence. This example avoids the effects of sloping, where the signal intensity decreases as the base length increases. In other words, by grouping the electrophoresis for each point mutation, for example, when the difference in base length of the stuffer sequence is 15 bp, the difference in base length within the point mutation can be suppressed to 15 bp×(3 bases−1)=30bp. This makes it possible to calculate the MT/WT more accurately in this example. In addition, in order to make the detection regarding the low frequency point mutation more advantageous, the method of making the PCR fragments related to the mutation shorter and performing electrophoresis earlier is effective.

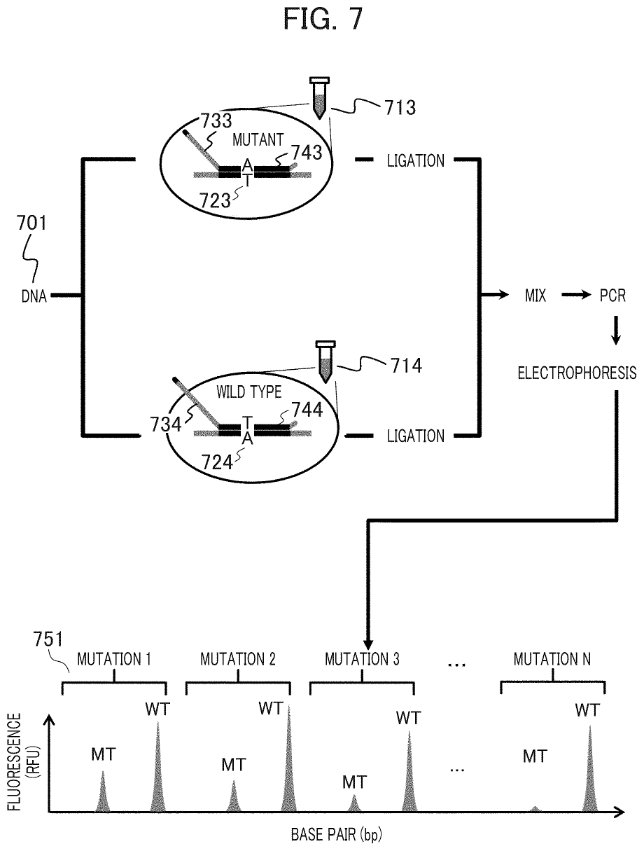

Next, referring to FIG. 7, a sixth example of the present invention will be described. FIG. 7 is an analysis reaction diagram of the sixth example of the present invention. This example limits the number of sample tubes to be divided to two. One sample tube 713 is assigned to the reaction of the typical point mutations to be detected, and another sample tube 714 is assigned to the reaction of Wild Type. The advantage of this method is that it reduces the reagent cost and effort required for the reaction by limiting the number of divided sample tubes to be divided to two. In addition, the base information on point mutations for LPOs 733 and 734 can be obtained from existing databases. In addition, since it is possible to avoid the competitive hybridization between the Wild Type LPO and the Mutant Type LPO, the rate MT/WT (the rate S_{MT}/S_{WT} of the intensity S_{MT} of the mutation-derived signal to the intensity S_{WT} of the wild-type derived signal) of the wild-type and mutant-type molecules present in the sample can be detected more accurately.

In this example, an initial DNA 701 is divided into two parts and added in equal amount to two different sample tubes 713 and 714. Different LPOs 733 and 734 are added to these. The 3′ ends of the LPOs 733 and 734 shown in the figure are adenine and thymine, respectively. In addition, the same RPOs 743 and 744 are added to respective tubes for the relevant point mutation. In this example, for the sake of simplicity, only one point mutation is shown, but the LPO and the RPO are reagents for probe groups that contain multiple point mutations. In addition, not all of the 3′ ends contained in the LPO 734 are thymine. The probe group selected for the 3′ end of the LPO 734 has a sequence complementary to the point mutation group corresponding to Wild Type. Therefore, at the 3′ end of each of the multiple LPOs used to detect Wild Type, Wild Type base sequence reflecting the point mutation information is arranged. These base sequences can be adenine, guanine, cytosine, or thymine.

By adjusting the length of the stuffer sequence, it is possible to design the base length of each probe. Therefore, the base length of the PCR product identified by electrophoresis can be easily sized, and it is possible to distinguish which peak is Wild Type and which peak is Mutant Type. Specifically, an electropherogram 751 shown in FIG. 7 can be obtained. As shown in the electropherogram 751, the MT/WT can be calculated for each Mutation 1, 2, 3 . . . N. In other words, it is possible to calculate the signals of point mutations and Wild Types from the assigned peaks and to quantify the MT/WT (quantify the rate S_{MT}/S_{WT} of the intensity S_{MT} of the mutation-derived signal to the intensity S_{WT} of the wild-type derived signal). In this example, for each point mutation, it is designed that the LPO 734 corresponding to Wild Type has a longer stuffer sequence than the LPO 733. In other words, a shorter stuffer sequence is assigned to the Mutant Type. The reason for this is that, in general, the abundance rate of the number of Mutant Types to the number of Wild Types is small. In particular, the amount of Mutant Types is small in the case of micromutations. On the other hand, it is known that in the capillary electrophoresis, the longer the length of the DNA fragment, the lower the amount of the fragment introduced into the capillary. Therefore, in order to detect even small amounts of micromutations as well as possible, the method of arranging the short stuffer sequences for point mutations is useful.

However, when there is an excessive amount of Wild Type, it is possible that it will cause background noise in the electrophoresis range with a molecular weight smaller than that of the Wild Type. In that case, it is useful to arrange a short stuffer sequence in the LPO 734 corresponding to the Wild Type, rather than the LPO 733.

It is also useful to compare Wild Type and Mutant Type more directly by grouping the electrophoresis for each point mutation.

When the difference in base length in the stuffer sequences is 15 bp, the difference in base length within the point mutations can be suppressed to 15 bp×(2 bases−1)=15 bp. This makes it possible to calculate the MT/WT more accurately in this example. This example also makes it possible to increase the number of genes with point mutations that can be detected in a single electrophoresis. This example shortens the base length of the low frequency point mutations and Wild Types to 15 bp, which has the effect of making the effect of sloping almost negligible.

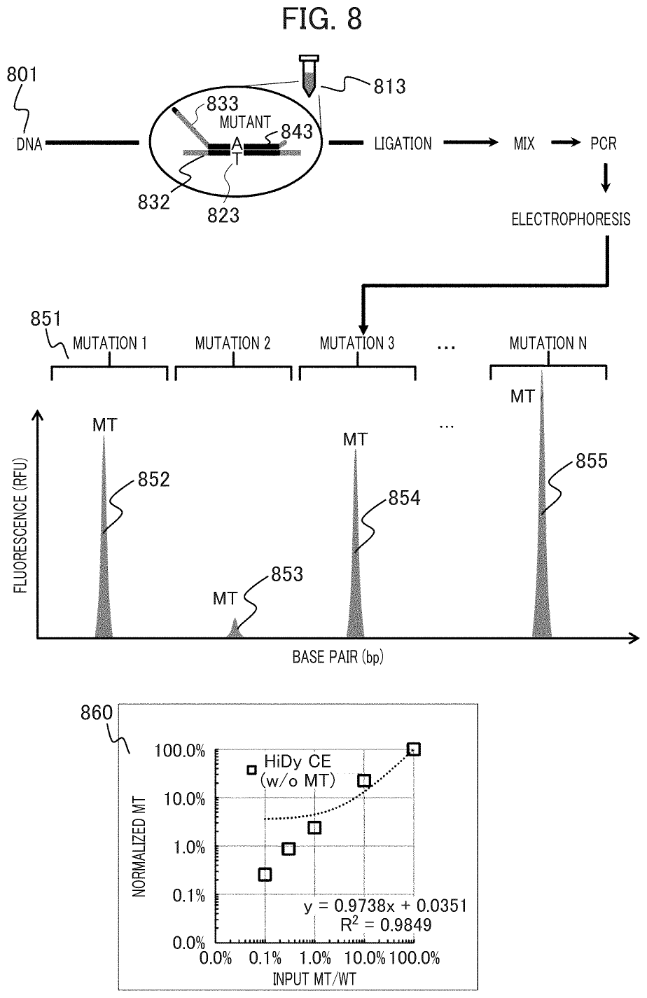

Next, referring to FIG. 8, a seventh example of the present invention will be described. FIG. 8 is an analysis reaction diagram of the seventh example of the present invention. This example describes a measurement method that detects only Mutant Type without using Wild Type.

Add 10 to 100 μg of a sample DNA 801 to a single sample tube 813. Add a LPO 833 and a RPO 843, which hybridize to a DNA target sequence 832 with point mutations in the DNA 801. The behavior shown here is for one point mutation sequence, but in actual reactions, there are multiple point mutation sequences in the DNA 801. This is just a description using one point mutation sequence as an example.

The 3′ end of the LPO 833 shown in the figure is adenine. In addition, the RPO 843 is added. The DNA target sequence 832 contains a point mutation thymine 823 as a point mutation thymine sequence. The point mutation thymine 823 forms a complementary strand with the adenine at the 3′ end of the LPO 833. Therefore, it is possible to ligate the gap between the LPO 833 and the RPO 843 using a ligase enzyme, and the LPO 833 and the RPO 843 form a single DNA strand.

In this example, for the sake of simplicity, only one point mutation is shown, but the LPO and the RPO are reagents for probe groups that contain multiple point mutations. Therefore, the 3′ end of the LPO 833 is not always adenine for all the point mutation sites, but any one of the four bases adenine, guanine, cytosine, or thymine is ligated to the 3′ end of each LPO 833, depending on the actual point mutation.

By adjusting the length of the stuffer sequence, it is possible to design the base length of each probe. Therefore, the base length of the PCR product identified by electrophoresis can be easily sized, and an electropherogram 851 shown in FIG. 8 can be obtained. As shown in the electropherogram 851, multiple peaks 852, 853, 854, and 855 can be obtained for each of Mutation 1, 2, 3 . . . N.

A graph 860 shown in FIG. 8 shows the normalized signal intensity of Mutations when the cell rate of the input Wild Type and Mutant Type cells is changed for one point mutation. The normalized signal intensity is calculated by dividing the signal volume of Mutant Type at different cell rates by the signal volume of Mutant Type when the rate of the Mutant Type is 100%, and then normalizing. The rate of Mutant Type in the cell and the signal volume from Mutant Type are in a proportional relationship. Therefore, when the signal intensity at 100% Mutant Type is measured in advance independently of the measurement by electrophoresis described above (when the maximum saturation value of the intensity S_{MT} of the mutation-derived signal is measured), the content rate (ratio) of mutant cells in the sample can be estimated from the signal intensity of point mutations measured in a certain experiment by comparing it with that value. In other words, the quantitative testing can also be performed in this example.

Note that this method cannot be achieved using conventional capillary sequencers. The reason for this is that when the cell rate is 100% Mutant Type-derived, the peak signal will cause saturation within the conventional dynamic range, making accurate measurement impossible. To be more specific, since the signal detected for a 1% point mutation is around 3,000 [ADU], the detected signal would be around 300,000 [ADU] for a 100% point mutation. Since the upper saturation limit that can be detected by a conventional capillary sequencer is 32,767 [ADU], the saturation occurs. Therefore, in order to perform the quantitative point mutation counting using the LPO and the RPO for Mutant Type, it is preferred to use a capillary sequencer with a high dynamic range. As an example of a high dynamic range, as described above, the upper limit is 200,000 [ADU] or more, and for example, 0 to 200,000 [ADU], but these are not limited to these values.

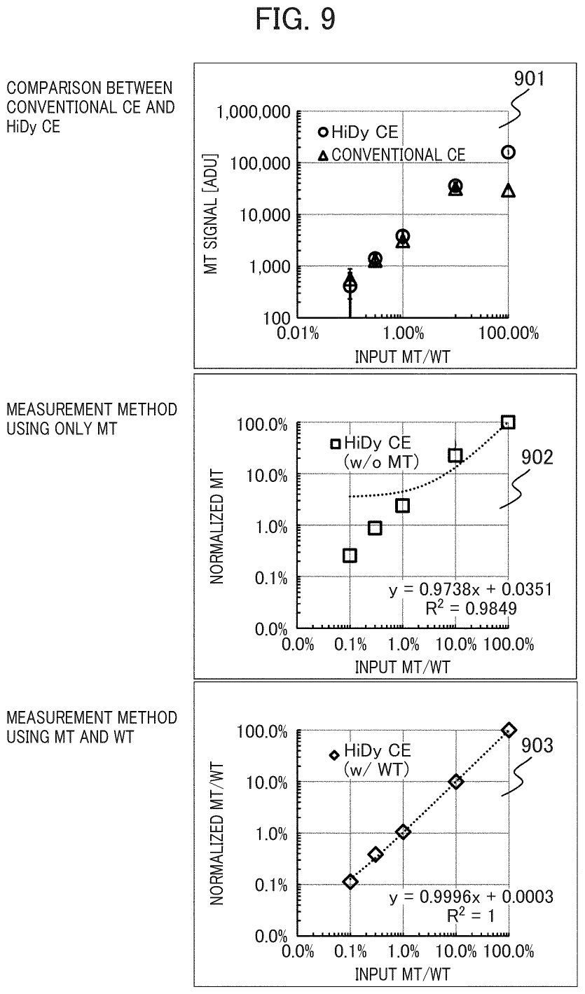

Next, referring to FIG. 9, an eighth example of the present invention will be described. FIG. 9 shows analytical reaction analysis results for the eighth example of the present invention.

In a graph 901 shown in FIG. 9, the horizontal axis of the graph shows rate of amount of Mutant Type to Wild Type, under the condition that the amount of Wild Type is fixed at 100 μg, for one point mutation. In addition, the vertical axis of the graph shows the signal intensity of the Mutant Type peak for the cell mix rate of MT/WT.

In the graph 901, it can be seen from the triangles in the figure that the signal is saturated in the cell rate MT/WT range from 10% to 100% in measurements performed using a conventional capillary sequencer (CE). In other words, when 100 ug of Mutant Type-derived DNA is reacted using a conventional capillary sequencer, the signal becomes saturated and the accurate measurement cannot be performed. On the other hand, as shown by the circles in the figure, with the HiDy capillary sequencer (HiDy CE) with a high dynamic range, it can be confirmed that the signal from the Mutant Type increases in proportion even when the cell rate MT/WT increases from 10% to 100%. Therefore, to detect mutations in a wide range from 0.1% to 100%, it is preferred to use the HiDy capillary sequence having a high dynamic range.

Next, a graph 902 shown in FIG. 9 will be described. Also in the graph 902, the horizontal axis of the graph shows the rate of the amount of Mutant Type to Wild Type is varied, under the condition that the amount of Wild Type is fixed at 100 μg, for one point mutation. In addition, the vertical axis of the graph is normalized by dividing the signal intensity of the peak of the Mutant Type for the cell mix rate of MT/WT by the signal value of the Mutant Type at 100% MT/WT.

The graph 902 shows the measurement results when only Mutant Type is targeted, and the LPO and the RPO probes for the Wild Type are not used, as described in the seventh example. Therefore, since there are no signals from the Wild Type, the normalization of the signals is performed by division within the Mutant Type. In the seventh example, there is a problem that the signal values of the Mutant Type with the MT/WT 100% used for normalization and the signal values of the Mutant Type for the different cell mix rate of MT/WT are separate electrophoresis, and thus, the variation in sample injections during electrophoresis cannot be corrected.

On the other hand, in a graph 903 shown in FIG. 9, the LPO and the RPO probes were arranged on the DNA target sequence, for Mutant Type and Wild Type in pairs for one point mutation, and the measurements were taken. The difference from the graph 902 is that during every electrophoresis, it is possible to simultaneously measure the signals from the Mutant Type and Wild Type.

The vertical axis of the graph in the graph 903 can be calculated for each electrophoresis by dividing the Mutant Type-derived signal by the Wild Type-derived signal. Therefore, there is the advantage that the variation in sample injection during electrophoresis can be corrected for each electrophoresis. This leads directly to improved measurement accuracy. The linearity is R²=0.9849 in the graph 902 while the linearity is R²=1 in the graph 903, indicating that the simultaneous measurement of Mutant Type and Wild Type is more accurate. In particular, as the rate of MT/WT decreases from 1% to 0.1%, the normalized signal value deviates from the ideal linear approximation line in the graph 902, whereas the graph 903 shows a better fit. This shows that the simultaneous measurement of signals from Mutant Type and Wild Type, even at low MT/WT, can provide more sensitive and reliable measurements. Therefore, it can be said that Mutant Type and Wild Type should be measured in pairs when detecting mutations using a capillary sequencer with a high dynamic range.

The above is a detailed description of a mutation rate detection method according to the present invention using examples (embodiments), but the present invention is not limited to the examples given above, and includes various variations. For example, the above-described embodiments have been described in detail in order to facilitate the understanding of the present invention, and the present invention is not necessarily limited to those including all of the described configurations. In addition, part of the configuration of one embodiment can be replaced with the configurations of other embodiments, and in addition, the configuration of the one embodiment can also be added with the configurations of other embodiments. In addition, part of the configuration of each of the embodiments can be subjected to addition, deletion, and replacement with respect to other configurations.

### LIST OF REFERENCE SIGNS

101, 102 Sequence
103 Point mutation guanine
104, 105 Sequence
106 Cytosine
107 Stuffer sequence
109, 110 Primer sequence
111 Stuffer sequence
112, 113 DNA target sequence
114 Normal base cytosine
115 to 117 PCR product
120 Electropherogram
201 Sample tube
202 to 205 LPO
206 RPO
212 to 215 Peak
222 to 225 DNA target sequence
250 Electropherogram
301 Sample tube
302 to 305 LPO
306 RPO
312 to 315 Peak
322 to 325 DNA target sequence
350 Electropherogram
401 Sample tube
402 to 405 LPO
406 RPO
412 to 415 Peak
422 to 425 DNA target sequence
350 Electropherogram
501 DNA
511 to 514 Sample tube
521 to 524 DNA target sequence
531 to 534 LPO
541 to 544 RPO
551 Electropherogram
601 DNA
612 to 614 Sample tubes
622 to 624 DNA target sequence
632 to 634 LPO
642 to 644 RPO
651 Electropherogram
701 DNA
713, 714 Sample tubes
733, 734 LPO
743, 744 RPO
751 Electropherogram
801 DNA
813 Sample tube
823 Point mutation thymine
832 DNA target sequence
833 LPO
843 RPO
851 Electropherogram
852 to 855 Peaks
860 Graph
901 to 903 Graphs

1.-6. (canceled)

7. A point mutation rate detection method used in a multiplex ligation-dependent probe amplification (MLPA) measurement, the point mutation rate detection method comprising:
a measurement step of measuring at least an intensity S_{MT} among the intensity S_{MT} of a mutation-derived signal as a fluorescence signal emitted from a mutation site of a sample and an intensity S_{WT} of a wild-type derived signal as a fluorescence signal emitted from a site of the sample other than the mutation site, using an electrophoresis device; and
a rate calculation step of calculating a rate of the S_{MT} to a reference value having a higher intensity than the S_{MT}, wherein
an upper limit of a dynamic range of the measurement for the fluorescence signal of the electrophoresis device is equal to or more than a predetermined value,
the reference value is a maximum saturation value of the S_{MT} measured independently of the measurement step in advance, and
the rate calculation step calculates the rate of the S_{MT} obtained in the measurement step from the maximum saturation value.

8. A point mutation rate detection method used in a multiplex ligation-dependent probe amplification (MLPA) measurement, the point mutation rate detection method comprising:
a measurement step of measuring an intensity S_{MT} of a mutation-derived signal as a fluorescence signal emitted from a mutation site of a sample and an intensity S_{WT} of a wild-type derived signal as a fluorescence signal emitted from a site of the sample other than the mutation site, using an electrophoresis device; and
a rate calculation step of calculating a rate of the S_{MT} to a reference value having a higher intensity than the S_{MT}, wherein
an upper limit of a dynamic range of the measurement for the fluorescence signal of the electrophoresis device is equal to or more than a predetermined value,
the reference value is the S_{WT} ,
the rate calculation step calculates a rate S_{MT}/S_{WT} of the S_{MT} to the S_{WT} from the S_{MT} and the S_{WT} obtained in the measurement step, and
the sample is divided into three or two parts in the MLPA measurement, one of the parts is used to generate the S_{WT} independently and selectively, and remaining two or one part is used to generate the S_{MT} independently and selectively.

9. The point mutation rate detection method according to claim 7, wherein
the upper limit of the dynamic range is 200,000 [ADU] or more, 400,000 [ADU] or more, 600,000 [ADU] or more, 800,000 [ADU] or more, or 1,000,000 [ADU] or more.

10. The point mutation rate detection method according to claim 7, wherein
the dynamic range is 0 to 200,000 [ADU], 0 to 400,000 [ADU], 0 to 600,000 [ADU], 0 to 800,000 [ADU], or 0 to 1,000,000 [ADU].

11. The point mutation rate detection method according to claim 8, wherein
the upper limit of the dynamic range is 200,000 [ADU] or more, 400,000 [ADU] or more, 600,000 [ADU] or more, 800,000 [ADU] or more, or 1,000,000 [ADU] or more.

12. The point mutation rate detection method according to claim 8, wherein the dynamic range is 0 to 200,000 [ADU], 0 to 400,000 [ADU], 0 to 600,000 [ADU], 0 to 800,000 [ADU], or 0 to 1,000,000 [ADU].
